(19) United States
(12) Patent Application Publication
(10) Pub. No.: US 2007/0141127 A1
(43) Pub. Date: Jun. 21, 2007

(54) Wet wipes with natural antimicrobial agents

(75) Inventors: Jorge Casas-Sanchez, El Prat De Llobregat (ES); Ralf Eduard Adam, Laudenbach (DE); Mathias Kurt Herrlein, Hofheim (DE); John Ernest Rice JR., West Chester, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/639,046

(22) Filed: Dec. 14, 2006

Related U.S. Application Data
(60) Provisional application No. 60/750,456, filed on Dec. 15, 2005.

(51) Int. Cl. A61K 8/89; A61K 9/70

(52) U.S. Cl. 424/443; 424/70.12

(57) ABSTRACT

Wet wipes impregnated with a composition having a pH value of between 2.5 and 5.0. The composition is in the form of an oil-in-water emulsion and comprises a non-ionic silicone surfactant, a natural antimicrobial active and a buffer system. The wipes may be used for personal cleansing, in particular as baby wipes.

### CROSS REFERENCE TO RELATED APPLICATION

This application claims the benefit of U.S. Provisional Application No. 60/750,456, filed Dec. 15, 2005, the substance of which is incorporated herein by reference.

### FIELD OF THE INVENTION

The invention relates to wet wipes which can be used for personal cleansing. The wipes are impregnated with a composition having a pH value of between about 2.5 and about 5.0. The composition is in the form of an oil-in-water emulsion and comprises a non-ionic silicone copolyol surfactant, a natural antimicrobial active and a buffer system.

### BACKGROUND OF THE INVENTION

Wet wipes have been commercially proposed for various uses. For example, it has been proposed to use wet wipes impregnated with a cleaning product for cleaning hard surfaces such as floors or kitchen surfaces. Wet wipes have also been used for personal cleansing, for example to remove woman's make-up or to clean and refresh the skin whilst traveling.

Wet wipes have also been proposed as a practical and hygienic tool to clean baby's skin, especially in the peri-anal area post defecation. Such wipes are practical because they are normally sold already impregnated with a baby lotion for cleaning and treating the baby's skin, and therefore do not require the parents to manipulate or travel with a bottle containing a baby lotion. These wipes are hygienic because they are disposable and are normally discarded after their first use.

Wet wipes are normally made of a porous or adsorbent sheet impregnated with a composition and packaged in an air-tight box or wrapper to avoid the loss of humidity or other volatile components during storage.

Antimicrobial wipes that are used to sterilize a surface normally comprise strong antimicrobial agents such as Triclosan (TCS) or Triclocarban (TCC). Even cleaning wet wipes that are not claiming disinfectant properties normally comprise a preservative system to prevent the proliferation of microorganisms such as bacteria, fungi or yeast during storage. Whilst required for safety reasons, these preservative systems typically comprise synthetic chemical compounds such as parabens. There is a growing demand from the public for a reduction of synthetic preservatives in personal care products. However reducing these synthetic preservatives would have a negative impact on microbiological safety requirements, so that there is an unmet need for a safe and effective preservative system with reduced amount or even no conventional synthetic chemical compounds and reduced irritation.

The pH of healthy skin has been assessed in the past to be between 4.5 and 6.0. Numerous products, in particular baby products, are formulated at a pH of about 5.5, as it is commonly believed that having personal care products matching the average pH of skin might be beneficial to skin's health, in particular maintaining the skin at its normal pH. Wet wipe products commercially available normally comprise a composition having a pH value above 5.0. As an exception, the inventors believe that a wet wipe product sold under the tradename “Soft & Gentle Baby Wipes by Seventh Generation Inc.” in the USA is impregnated with a composition having a measured pH of ca. 3.3. This product comprises potassium sorbate as preservative and does not comprise a natural antibacterial agent or a dimethicone copolyol surfactant.

Whilst the pH of the composition is an important factor for the performance of the wet wipes, the inventors also believe that the buffer capacity of the composition is an important characteristic in order to maintain or bring back the skin to its natural pH. On skin, any product application competes with the buffer system of the skin (e.g. amino acids, breakdown products of lipids, urocanic acids). The skin of babies is often exposed to feces and urine residues. This mix has been shown to push up the pH on the baby's skin above the natural values of pH.

The inventors have found that the composition used in wet wipes could advantageously have a pH of below 5.0, and feature a buffer system. In these conditions, it was found that synthetic preservative agents could be partially or even completely replaced by natural antimicrobial actives. The inventors also believe that these conditions make the wet wipes of the invention particularly suitable for cleaning bodily waste on the body, in particular the wipes of the invention may be able to sustainably bring back the pH of the skin to physiological values (around pH 5.5).

U.S. Pat. No. 4,732,797 and U.S. Pat. No. 4,772,501 discloses a wet wiper product of the type having a fibrous wipe, a liquid preservative composition, and an enclosure for the fibrous wipe and the liquid preservative composition. The liquid preservative composition consists essentially of: (a) a mixture of citric acid and sorbic acid as the preservative component; (b) water; and (c) optional ingredients selected from the group consisting of skin moisturizers and fragrance compounds.

U.S. Pat. No. 5,141,803 discloses a nonwoven wipe impregnated with an aqueous composition having a pH of from 3.5 to 4.5 and containing a preservative system comprising potassium sorbate, citric acid, disodium EDTA and a cationic biocide selected from polyhexamethylene biguanide hydrochloride and poly[oxyethylene(dimethyliminio)ethylene(dimethyliminio)ethylene dichloride].

WO03/079794A1 relates to the use of olive tree extracts in detergent, rinsing agents and cleansing agents.

WO05/009405A1 discloses wet wipes comprising a Yucca species extract as pathogen selective antimicrobial agent and an optional broad spectrum antimicrobial agent.

EP516,547, EP154,837 and US01/0046507 independently describe cosmetic emulsions of the oil-in-water type consisting of an aqueous phase, an oily phase and an dimethicone copolyol surfactant.

WO98/55096 discloses antimicrobial wipes comprising a porous or absorbent sheet impregnated with an antimicrobial cleansing composition. The antimicrobial cleansing composition comprises an antimicrobial active; an anionic surfactant; a proton donating agent and water. The composition is adjusted to a pH of from about 3.0 to about 6.0.

U.S. Pat. No. 6,287,583 discloses a low-pH, acid-containing personal care compositions comprising from about 0.5% to about 80% of a surfactant, from about 0.1% to about 12% of a polymeric acid; and from about 3% to about 98.899% of water, wherein the composition is adjusted to a pH of from about 3.0 to about 6.0. The composition may be used on a wipe.

Whilst the documents of the prior art individually disclose some of the elements of the present invention, they fail to teach the combination of all the elements of the wet wipes claimed. The inventors have found that the combination of the elements claimed allows reducing the levels of conventional synthetic preservatives in a wet wipe whilst still preventing the growth of harmful microorganism during storage, provides excellent skin feel and reduced skin irritation, and may help returning a soiled skin's pH to a normal value. The present wet wipes are especially suitable to be used as baby wipe, as moistened toilet papers for adults or feminine care hygiene wipes.

### SUMMARY OF THE INVENTION

The present invention is directed to a wet wipe comprising a substrate impregnated with a composition. The composition has a pH of between about 2.5 and about 5.0, and comprises:

i) from 0.01 to 5% of a dimethicone copolyol surfactant,

ii) a natural antibacterial agent,

iii) a buffer system having a minimum Buffer Capacity.

While the specification concludes with claims which particularly point out and distinctly claim the invention, it is believed the present invention will be better understood from the following description.

### BRIEF DESCRIPTION OF THE DRAWING

FIG. 1 shows a titration curve for three different compositions.

### DETAILED DESCRIPTION OF THE INVENTION

All percentages are by weight of total composition unless specifically stated otherwise. All ratios are weight ratios unless specifically stated otherwise.

All documents cited are, in relevant part, incorporated herein by reference; the citation of any document is not to be construed as an admission that it is prior art with respect to the present invention.

Wet Wipes

Wet wipes or wet-tissues are the general terms to describe a substrate, generally a non-woven material, impregnated with a composition comprising water or another liquid solvent (e.g. ethanol). Whilst not limited to a particular use, the wet wipes of the invention may be intended for the cleaning of the body, in particular the peri-anal area after defecation and/or the external genital area after urination.

Wet wipes are generally of sufficient dimension to allow for convenient handling while being small enough to be easily disposed of by the sewage system or discretely disposed of in garbage bins. The substrate making of the wipes is generally soft and flexible, potentially having a structured surface enhancing its cleaning performance. The substrate is often a non-woven material, generally made of synthetic compounds. However, woven materials as well are also within the scope of the present invention.

In one embodiment of the present invention, the non-woven substrate may comprise fibers made from a material selected from the group consisting of polyolefins, polyesters, cellulose, cellulose derivatives, rayon, polyamides, polyesteramides, polyvinyl alcohols, cotton, and combinations thereof. The substrate usable for this invention can be manufactured via any suitable process, such as but not limited to, spunlace process. The substrate may have a dry basis weight of between about 40 grams per square meter (g/m²) and about 75 g/m², more precisely between about 45 g/m² and about 65 g/m². Various examples of suitable substrates are disclosed in the patent literature, see for example U.S. Pat. No. 6,960,349B2, col. 10 line 24 to col. 11 line 39.

The wipes of the invention are impregnated with an oil-in-water emulsion composition, which may be intended to facilitate skin cleaning, provide a smooth feeling and help the skin returning to its normal pH. Generally the composition is of sufficiently low viscosity to impregnate the entire structure of the wipe. In some other instances, the composition can be primarily present at the wipe surface and to a lesser extent in the inner structure of the wipe. In one optional embodiment the composition is releasably carried by the material, that is, the composition is contained either in or on a substrate and is readily releasable from the substrate by applying some force to the substrate, for example, wringing the substrate, or wiping a surface, such as a child's bottom, with the wet-wipe.

The amount of composition applied on or impregnated into the substrate can vary. Usually, the amount of the composition present on the substrate per area of surface is between 10% and 1000% of the basis weight of the substrate.

The composition impregnating the wet wipe of the present invention comprises water. The amount of water of the impregnating composition is normally, but not limited to, at least about 80 percent by weight water, and may be at least about 85, 90 or 95 percent by weight water.

Dimethicone Copolyol Surfactant

The dimethicone copolyol surfactant used in the invention is a nonionic polysiloxane copolymer having emulsifying ability, and may comprise a methylpolysiloxane moiety and a poly(oxyalkylene)methylpolysiloxane moiety. The dimethicone copolyol is present in amount of from 0.01% to 5% by weight of the composition, preferably in a mount of from 0.1% to 2%.

The dimethicone copolyol surfactant may have an HLB value of from about 4 to about 17, and a molecular weight of from about 10,000 to about 20,000. The HLB value is a theoretical index value which describes the hydrophilicity-hydrophobicity balance of a specific compound. Generally, it is recognized that the HLB index ranges from 0 (very hydrophobic) to 40 (very hydrophilic). The HLB value of the lipophilic surfactants may be found in tables and charts known in the art, or may be calculated with the following general equation: HLB=7+(hydrophobic group values)+(hydrophilic group values). The HLB and methods for calculating the HLB of a compound are explained in detail in “Surfactant Science Series, Vol. 1: Nonionic Surfactants”, pp 606-13, M. J. Schick (Marcel Dekker Inc., New York, 1966). The following are non-exhaustive examples of suitable dimethicone copolyol surfactants.

Suitable dimethicone copolyol surfactants include those having the following general formula (I):

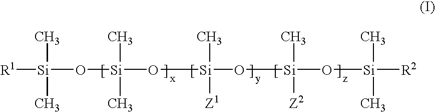

wherein Z¹, R¹ and R² are independently —(CH₂)ₜ—O(C₂H₄O)ₚ(C₃H₆O)_{q}R³,

R³is H, CH₃, or CH₂CH₃,

p is from 0 to about 50,

q is from 0 to about 30, wherein p and q are not 0 at the same time;

t is 0, 1, 2, 3 or 4;

x is from 1 to about 250,

y is from 1 to about 40, and

z is from 0 to about 100, and Z² is an alkyl group having from about 10 to about 22 carbons, preferably from about 16 to about 18 carbons.

Suitable dimethicone copolyol surfactants include those having the following formula (H):
R¹(CH₃)₂SiO—[(CH₃)₂SiO]ₙ—Si(CH₃)₂R², (II)

wherein

n=50 to 250,

R¹, R²=—(CH₂)ₘ—O—(C₂H₄O)ₓ—(C₃H₆O)_{y}R³, wherein R¹ and R² may be the same or different,

m=2 to 4,

x=3 to 100,

y=0 to 50,

R³═H, CH₃, or CH₂CH₃.

Examples of compounds according to formula (II) are described in US2005/0031560 (assigned to the Procter & Gamble Company).

Suitable dimethicone copolyol surfactants include those comprising an alkylpolysiloxane moiety, for example, but not limited to those having the following formula (III):

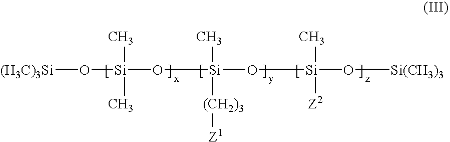

wherein Z¹ is —O(C₂H₄O)ₚ(C₃H₆O)_{q}H,

p is from 0 to about 50,

q is from 0 to about 30, wherein p and q are not 0 at the same time;

x is from 1 to about 200,

y is from 1 to about 40, and

z is from 1 to about 100,

and Z² is an alkyl group having from about 10 to about 22 carbons, preferably from about 16 to about 18 carbons.

Examples of alkyldimethicone copolyol surfactant compounds according to formula (III) can be found in US2004/0219122 (assigned to the Procter & Gamble Company), and include cetyl dimethicone copolyol and stearyl dimethicone copolyol. A commercially available alkyldimethicone copolyol is available from Goldschmidt Personal Care under the tradename ABIL EM-90 (CTFA name: Cetyl PEG/PPG-10/1 Dimethicone polysiloxane) and has an HLB of about 5 and a molecular weight of about 13,000.

Other suitable dimethicone copolyol surfactants commercially available include, but are not limited to, the following products (tradename in brackets):
products available from GE Silicones: Bis-phenylpropyldimethicone (SF1555), PEG-12 Dimethicone (SF 1288), PEG-17 Dimethicone (Silsoft 895), PPG-17 Dimethicone (Silsoft 900,920,940), PEG/PPG-8/26-Dimethicone (Silsoft 400, 405), PEG/PPG-20/15-Dimethicone (SF1188A, Silsoft 445), PEG/PPG-20/23-Dimethicone (Silsoft 430, 440), PEG/PPG-23/6-Dimethicone (Silsoft 475);
products available from Dow Corning: PEG-12 Dimethicone (DC193 Surfactant), PEG/PPG-15/15-Dimethicone (DC5330 Fluid), PEG/PPG-17/18-Dimethicone (DC 2-5220 Resin Modif.), PEG/PPG-18/18-Dimethicone (DC 190 Surfactant), PEG/PPG-30/10-Dimethicone (BY25-339), PPG-2-Dimethicone (DC1248 fluid);
products available from Degussa: Bis-PEG/PPG-16/16-PEG/PPG-16/16-Dimethicone (Abil Care 85), Bis-PEG/PPG-20/20-Dimethicone (Abil B8832), PEG-14-Dimethicone (Abil B8843), PEG/PPG-4/12-Dimethicone (Abil B8852), PEG/PPG-20/6-Dimethicone (Abil B88183), PEG/PPG-14/4-Dimethicone (Abil B8851), PEG/PPG-20/20-Dimethicone (Abil B8851)
product available from Wacker: PEG/PPG-25/25-Dimethicone (Belsil DMC6031).
   Natural Antibacterial Agent

The composition used in the wet wipes of the invention comprises at least one natural antibacterial agent extracted from a natural source, normally a plant. By “natural” we mean a product that was extracted from a living organism. Natural products are normally a mixture of many chemical compounds with various activities. The term “natural”, as used herein, therefore do not encompass synthetically produced compound, which are normally pure, even if these synthetic products have been linked to the antibacterial property of certain natural antibacterial ingredients. The “term” natural as used herein however encompasses preparations that have been concentrated, purified or chemically treated after a first extraction from a living source.

Natural antibacterial agents may be extracted from plants using various methods, for example steam distillation, simple distillation or mechanical extraction, and the resulting preparations are variously referred as decoction, dry preparation, extract (concentrated, dry, liquid or soft), oils (essential, fixed, infused), powder or tincture, depending on the method used and the form of the preparation obtained. It is known that the natural antibacterial agent can be obtained from various parts of the plant, such as the fruits, leaves, bark or roots.

Typical natural antibacterial agents include those extracted from the following plants, especially but not limited to their essential oils: anise, olive, lemon, orange, rosemary, wintergreen, thyme, lavender, cloves, hops, tea tree, citronella, wheat, barley, lemongrass, cedar leaf, cedarwood, cinnamon, fleagrass, geranium, sandalwood, violet, cranberry, eucalyptus, vervain, peppermint, ylang-ylang, gum benzoin, basil, sea-buckthorn, grapefruit, fennel, fir and balsam.

Among these plants, we can preferably include, but not limit to, the group consisting of olive tree, rosemary, white and green tea, balm mint, chamomile and liquorice and their mixtures.

It has also been suggested that some animals, especially marine animals such as certain sponges, certain marine snails, certain mussels, may naturally produce biologically active agents with anti-microbial effects. Chitosan is a linear polysaccharide extracted from chitin, which is the structural element in the exoskeleton of crustaceans (crabs, shrimp, etc.), and has been proven to show antibacterial activity (see “Antimicrobial Finishing of Polypropylene Nonwoven Fabric by Treatment with Chitosan Oligomer”, Journal of Applied Polymer Science, Vol. 74, 2911-2916 (1999), Shin, You, Min.

The required amount of natural antibacterial agents in the composition may vary depending on factors such as their anti-microbial activity, the concentration in actives of the preparation used, the strength of the preservative effect sought, and the presence or not of conventional chemical preservatives. It may be suitable to include natural antimicrobial agent preparations starting at 0.01% by weight of the composition to deliver antimicrobial properties, although this indicative value is not limiting the present claims.

Buffer System—pH Adjustement

The composition used in the invention comprises a buffer system in order to stabilize the pH of the composition, in particular to make the pH resistant to change when contacted with the skin. Body wastes such as urine and fecal matter can raise the pH of the skin above its natural value. The initial pH of the composition is between about 2.5 and about 5.0, which was found to enhance the preservative properties of natural antimicrobial, and at the same sufficiently low to counteract the effect of body wastes on pH, thus helping the pH of the skin recover its normal value after soiling. Furthermore the composition impregnating the wipes comprise a buffer system, so that the wet wipes can be used to clean a relatively high amount of bodily waste, as would be found post-defecation or post-urination in babies or adults.

The approach of the inventors is meant to support and stabilize the physiological skin pH and not to compete with it. As such, the invention is intended to minimize negative external impact on the natural skin pH, e.g. by urine and feces mix.

Buffer systems are known in the art and usually consist of either a weak acid and its salt or a weak base. The resistive action is the result of the equilibrium which is set up between the weak acid and the salt or weak base.

The following acids (and their conjugate bases) are non limiting examples of acids which can be used to form the buffer system of the invention: adipic acid, tartaric acid, citric acid, maleic acid, malic acid, succinic acid, glycolic acid, glutaric acid, benzoic acid, malonic acid, salicylic acid, gluconic acid, polymeric acids, phosphoric acid, sorbic acid, carbonic acid, fumaric acid, ascorbic acid, phtalic acid, their salts, and mixtures thereof.

Suitable polymeric acids can include homopolymers, copolymers and terpolymers, and may contain at least 30 mole % carboxylic acid groups. Specific examples of suitable polymeric acids useful herein include straight-chain poly(acrylic) acid and its copolymers, both ionic and nonionic, (e.g., maleic-acrylic, sulfonic-acrylic, and styrene-acrylic copolymers), those cross-linked polyacrylic acids having a molecular weight of less than about 250,000, preferably less than about 100,000 poly (α-hydroxy) acids, poly (methacrylic) acid, and naturally occurring polymeric acids such as carageenic acid, carboxy methyl cellulose, and alginic acid.

A buffer system can be characterized by the Buffer Capacity it provides to the composition. Buffer Capacity is defined herein as the volume of sodium hydroxide (NaOH) 0.1N required for raising the pH of a volume of 50 ml of the composition from its initial value pH (which is below 5.5) to a pH of 6.5 at 21° C. The Buffer Capacity can be either calculated (for example with a computer program or by hand) when the components of the composition are known using the pKa values of the acid and conjugate bases present, or it can also be determined experimentally through a simple acid-base titration using sodium hydroxide. The Buffer Capacity of the compositions of the invention is of at least about 10 ml NaOH 0.1N. The Buffer Capacity may also have higher value, such as at least about 15 ml, 20 ml, 25 ml, 30 ml or 35 ml. Higher Buffer Capacity for the composition may improve the performance of the wipes at higher level of soiling.

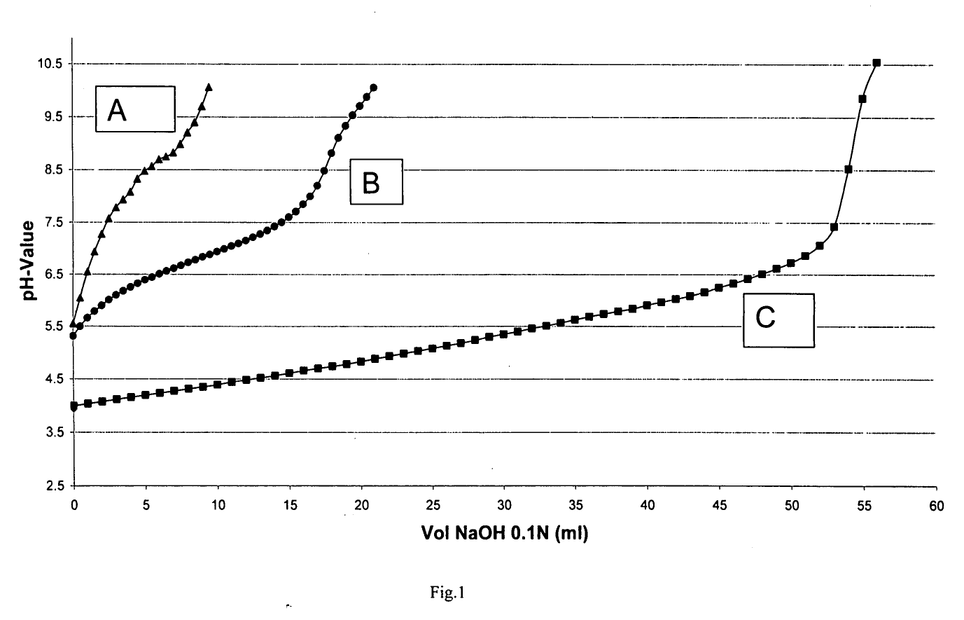

FIG. 1 shows a titration curve for three compositions (A, B, C) used to impregnate wet wipes. Composition A is a composition formulated without a buffer system, and the pH of the composition raises very quickly. Composition B comprises a weak buffer system (sodiumdihydrogenphosphate dihydrate 0.35%), and the pH raises moderately quickly. Composition C comprises a buffer system according to the present invention (in this case citric acid 0.53%, trisodium citrate 0.30% and benzoic acid 0.05%, sodium benzoate 0.25%).

The initial pH of the composition of the invention is adjusted to a value between about 2.5 and about 5.0. It may be preferred to have an initial pH of at least 3.5 as at a lower value the composition may be irritating. The initial pH of the composition is preferably from about 3.0 to about 4.9, more preferably from about 3.5 to about 4.5. The desired initial pH can be provided by the buffer system itself, wherein in this case the non-exclusive list of examples of organic acids discussed above may be used (adipic acid, tartaric acid, citric acid, maleic acid, malic acid, succinic acid, glycolic acid, glutaric acid, benzoic acid, malonic acid, salicylic acid, gluconic acid, polymeric acids, phosphoric acid, sorbic acid, carbonic acid, fumaric acid, ascorbic acid, phtalic acid, their salts, and mixtures thereof). In case a lower initial pH than the pH provided by the buffer system is required, additional amount of mineral acid may be added to the composition. A non-exclusive list of examples of mineral acid for use herein are hydrochloric, phosphoric, sulfuric and mixtures thereof. By “initial pH” we mean the pH of the composition before the wipe has been used. This initial pH may be experimentally measured on the composition itself just before the composition is added or impregnated into the substrate at the point of manufacture. Alternatively, the composition may be squeezed out of already manufactured wet wipes, before they have been used, to make the pH measurement.

Other Ingredients

The compositions of the invention may comprise as additional ingredients any of the ingredients normally used in wet wipes. The CTFA International Cosmetic Ingredient Dictionary, Eleventh Edition, describes a wide variety of non-limiting cosmetic and pharmaceutical ingredients commonly used in the skin care industry, which are generally suitable for use in the compositions of the present invention. Non-limiting examples of functional classes of ingredients are described in this reference. Examples of these functional classes include: antioxidants, binders, biological additives, bulking agents, chelating agents, colorants, cosmetic astringents, cosmetic biocides, denaturants, drug astringents, emulsifiers, external analgesics, film formers, fragrance components, humectants, opacifying agents, plasticizers, preservatives, skin-conditioning agents (emollient, humectants, miscellaneous, and occlusive), skin protectants, solubilizing agents, suspending agents (non-surfactant), sunscreen agents, perfumes, ultraviolet light absorbers, vitamins, perfumes, and viscosity increasing agents (aqueous and non-aqueous, for example xanthan gum).

In particular, the compositions may advantageously comprise a skin conditioning agent, such as an emollient. Emollients are cosmetic ingredients which help to maintain the soft, smooth, and pliable appearance of skin. A suitable, non-limiting, lipophilic emollient is caprylic capric triglyceride. A detailed list of suitable lipophilic skin conditioning agents can be found on U.S. Pat. No. 6,287,583. These lipophilic skin conditioning agents are suitable to form partially or totally the internal (oil) phase of the oil-in-water emulsion. Hydrophilic ingredients are also suitable, for example glycerine.

The composition may contain reduced or no amount of synthetic preservative ingredients such as parabens, and reduced or no amount of synthetic antibacterial agents such as Triclosan (5-Chloro-2-(2,4-Dichlorophenoxy)phenol) or (Triclocarban N-(4-Chlorophenyl)-N′-(3,4-Dichlorophenyl)Urea).

For safety reasons, in particular if only low levels of natural antimicrobial actives are included, it may still be required to include in the composition some amount of conventional chemical preservatives, in that case it is preferred to use preservatives selected from the following list: benzyl alcohol, 3-acetyl-6-methylpyran-2,4(3H)-dione (dehydroacetic acid) and its salts, 4-hydroxybenzoic acid its salts and esters, sorbic acid (hexa-2,4-dienoic acid) and its salts, propionic acid and its salts, poly (1-hexamethylenebiguanide) hydrochloride, benzoic acid, phenoxyethanol, its salts and esters, and mixtures thereof.

### EXAMPLES

The examples below disclose formulations that can be used as the composition within the present invention. These compositions can be applied on or impregnated into any suitable substrate, such as those discussed above. For example, the compositions may be applied on a substrate made of 60% (w/w) polypropylene fibers and 40% (w/w) viscose fibers, and having an average fiber lengths being about 38 mm to 40 mm (available for example from PGI, USA), to prepare a wet-wipe in accordance with the present invention.

The ingredients of the examples may be processed according to the following instructions. A mixing time of about 2 min is allowed in between the process steps:

a. water is added into the vessel. Agitation is started and maintained through the whole process;

b. water soluble-ingredients (i.e. EDTA, sodium benzoate) are delivered and stirred for 15 min with a propeller stirrer;

c. xantham gum (when present) is added; the dimethicone copolyol surfactant and the caprylic capric triglyceride (when present) are mixed together and the resulting composition is added into the vessel while stirring is increased at 220 rpm (these ingredients may be bought already premixed, as in Abil® Care 85). An extra 10 min of stirring time is allowed for good dispersion. A recirculation loop may be used with the static mixer at this stage to improve dissolution and mixing;

e. the preservative ingredients (either separately or premixed) are added into the vessel when present. An extra 3 min of stirring time is allowed;

f. the solubilizer-surfactant (i.e. Emulsogen) is added into the vessel;

g. the natural ingredients are added into the vessel. An extra 2 min of stirring time is allowed for good dispersion;

h. the buffer system (e.g. citric acid and sodium citrate) is added into the vessel. An extra stirring time of 15 min is allowed before end of batch;

i. the composition can be discharged for further processing.

### Example 1

| INCI | TRADE NAME | % w/w [g] |
|---|---|---|
| Aqua | Water | QS |
| Disodium EDTA | EDTA BD | 0.1 |
| Sodium benzoate | Sodium benzoate | 0.20 |
| Xanthan Gum | Keltrol | 0.15 |
| PEG-40 Hydrogenated Castor Oil | Emulsogen HCW 049 | 0.99 |
| PEG/PPG-25/25-Dimethicone | Belsil DMC6031 | 0.50 |
| Caprylic capric triglyceride | Tegosoft CT | 0.08 |
| Phenoxyethanol, Benzoic Acid, | TPI 1824 | 0.6 |
| Dehydroacetic Acid | | |
| Olive tree leaf extract | Eurol BT | 0.04 |
| Citric Acid | Citric Acid | 0.29 |
| Trisodium Citrate | Trisodium Citrate | 0.16 |

### Example 2

| INCI | TRADE NAME | % w/w [g] |
|---|---|---|
| Aqua | Water | QS |
| Disodium EDTA | EDTA BD | 0.1 |
| Sodium benzoate | Sodium benzoate | 0.10 |
| Xanthan Gum | Keltrol | 0.20 |
| PEG-40 Hydrogenated Castor Oil | Emulsogen HCW 049 | 1.10 |
| Bis-PEG/PPG-16/16 PEG/PPG | Abil Care 85 | 0.30 |
| Dimethicone And Caprylic capric | | |
| triglyceride | | |
| Phenoxyethanol, Benzoic Acid, | TPI 1824 | 0.7 |
| Dehydroacetic Acid | | |
| Rosemary (Rosemarinus officinalis | Rosemary oil | 0.2 |
| oil) | | |
| Citric Acid | Citric Acid | 0.50 |
| Trisodium Citrate | Trisodium Citrate | 0.35 |

### Example 3

| INCI | TRADE NAME | % w/w [g] |
|---|---|---|
| Aqua | Water | QS |
| Disodium EDTA | EDTA BD | 0.1 |
| Aloe Barbadensis | Ritaloe | 0.025 |
| Sodium Benzoate | Sodium Benzoate | 0.25 |
| Xanthan Gum | Keltrol | 0.18 |
| Bis-PEG/PPG-16/16 PEG/PPG- | Abil Care 85 | 0.2 |
| 16/16 Dimethicone, Caprylic/Capric | | |
| Triglyceride | | |
| PEG-40 Hydrogenated Castor oil | Emulsogen HCW 049 | 0.6 |
| Benzylalcohol | Benzylalcohol | 0.5 |
| Dehydroacetic acid | Dehydroacetic acid | 0.06 |
| Bisabolol, Caprylic Capric | Chamomile extract | 0.03 |
| Triglycerides, Chamomilla Recutita, | | |
| Matricaria Flower Extracts | | |
| Propylene Glycol, | Extrapone Olive Leaf | 0.8 |
| Phenoxypropanediol, Olea Europaea | | |
| (Olive) Leaf Extract, Maltodextrin | | |
| Citric acid | Citric acid | 0.40 |
| Trisodium Citrate | Trisodium Citrate | 0.25 |

### Example 4

| INCI | TRADE NAME | % w/w [g] |
|---|---|---|
| Aqua | Water | QS |
| Disodium EDTA | EDTA BD | 0.1 |
| Aloe Barbadensis | Ritaloe | 0.0025 |
| Sodium Benzoate | Sodium benzoate | 0.2 |
| Xanthan Gum | Keltrol | 0.22 |
| PEG-40 Hydrogenated Castor Oil | Emulsogen HCW 049 | 0.88 |
| Bis-PEG/PPG-16/16 PEG/PPG | Abil Care 85 | 0.55 |
| Dimethicone Caprylic capric | | |
| triglyceride | | |
| Phenoxyethanol, Benzoic Acid, | TPI 1824 | 0.5 |
| Dehydroacetic Acid | | |
| Bisabolol, Caprylic Capric | Chamomile Extract | 0.03 |
| Triglycerides, Chamomilla Recutita, | | |
| Matricaria Flower Extracts | | |
| Citric Acid | Citric Acid | 0.53 |
| Trisodium Citrate | Trisodium Citrate | 0.33 |
| Propylene Glycol, Rosmarinus | Extrapone ® | 0.5 |
| Officinalis (Rosemary) Leaf Extract | Rosemary Leaves | |
| Propylene Glycol, | Extrapone ® Olive | 0.5 |
| Phenoxypropanediol, Olea Europaea | Leaves | |
| (Olive) Leaf Extract, Maltodextrin | | |
| Glycerine | Glycerine | 3.0 |

Manufacture

The step of providing the composition onto the wipes substrate can be achieved by any conventional application process, such as (but not limited to) spraying, printing, dipping, and coating (for example with the use of a curtain coater or a slot coater).

Article of Commerce

The wet wipes of the present invention may be sold to the user in a container to form an article of commerce. The article of commerce of the present invention typically comprises (a) a container, and (b) at least one, usually several, wet wipe as described above.

Containers useful in the present article include but are not limited, for example, PET tubs, flow wrap pouches, precut sachets for individually packed wet wipes, and other packaging known in the art as suitable for wet wipes. The container may be air tight so that the wet wipes do not dry during storage.

The container can be made of any suitable material or materials, and can be manufactured in any suitable manner. For example, the container can be made of polystyrene, polypropylene, PET, POET, polyethylene, polyester, polyvinyl alcohol, or the like. The containers may also be made of mixtures of materials. The containers can be manufactured by, for example, a vacuum molding process or an injection molding process, or any suitable process.

The articles of commerce may be intended (for example through packaging labelling or advertising) for general purpose cleaning (including inanimate surfaces), for personal cleaning, in particular as baby wipes for baby cleaning (the term baby as used herein covers toddler until the age of about 6) or as toilet paper. The wet wipes of the invention may also be used by carers in charge of incontinent adults which are not self-sufficient.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A wet wipe comprising a substrate impregnated with a composition,
wherein the composition is an oil-in-water emulsion and the composition has a pH of between 2.5 and 5.0, and
wherein the composition comprises:
i) from 0.01 to 5% of a dimethicone copolyol surfactant,
ii) a natural antibacterial agent,
iii) a buffer system providing the composition with a Buffer Capacity of at least 10 ml NaOH 0.1N as measured by the test described herein.

2. The wet wipe of claim 1, wherein the dimethicone copolyol surfactant has the following formula: 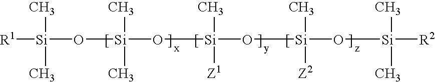
wherein Z¹, R¹ and R² are independently —CH₂)ₜ—O(C₂H₄O)ₚ(C₃H₆O)_{q}R³,
R³ is H, CH₃, or CH₂CH₃.
t is 0, 1, 2, 3 or 4,
p is from 0 to about 50,
q is from 0 to about 30, wherein p and q are not 0 at the same time;
x is from 1 to about 250,
y is from 1 to about 40, and
z is from 0 to about 100, and Z² is an alkyl group having from about 10 to about 22 carbons, preferably from about 16 to about 18 carbons.

3. The wet wipe of claim 1, wherein the dimethicone copolyol surfactant is selected from Bis-phenylpropyldimethicone, PEG-12 Dimethicone, PEG-17 Dimethicone, PPG-17 Dimethicone, PEG/PPG-8/26-Dimethicone, PEG/PPG-20/15-Dimethicone, PEG/PPG-20/23-Dimethicone, PEG/PPG-23/6-Dimethicone, PEG-12 Dimethicone, PEG/PPG-15/15-Dimethicone, PEG/PPG-17/18-Dimethicone, PEG/PPG-18/18-Dimethicone, PEG/PPG-30/10-Dimethicone, PPG-2-Dimethicone, Bis-PEG/PPG-16/16-PEG/PPG-16/16-Dimethicone, Bis-PEG/PPG-20/20-Dimethicone, PEG-14-Dimethicone, PEG/PPG-4/12-Dimethicone, PEG/PPG-20/6-Dimethicone, PEG/PPG-14/4-Dimethicone, PEG/PPG-20/20-Dimethicone, PEG/PPG-25/25-Dimethicone, Cetyl Dimethicone Copolyol, Stearyl Dimethicone Copolyol, Cetyl PEG/PPG-10/1 Dimethicone polysiloxane and mixtures thereof, preferably wherein dimethicone copolyol surfactant is Bis-PEG/PPG-16/16 PEG/PPG-16/16 Dimethicone.

4. The wet wipe of claim 1, wherein the buffer system comprises an ingredient selected from the group consisting of adipic acid, tartaric acid, citric acid, maleic acid, malic acid, succinic acid, glycolic acid, glutaric acid, benzoic acid, malonic acid, salicylic acid, gluconic acid, polymeric acids, phosphoric acid, sorbic acid, carbonic acid, fumaric acid, ascorbic acid, phtalic acid, their salts, and mixtures thereof, preferably wherein the composition comprises a mixture of citric acid and citrate salt.

5. The wet wipe of claim 1, wherein the pH of the composition is at least 3.5.

6. The wet wipe of claim 1, wherein the natural antibacterial agent comprises an extract from a plant selected from the group consisting of anise, olive, lemon, orange, rosemary, wintergreen, thyme, lavender, cloves, hops, tea tree, citronella, wheat, barley, lemongrass, cedar leaf, cedarwood, cinnamon, fleagrass, geranium, sea-buckthorn, grapefruit, ylang-ylang, sandalwood, violet, cranberry, eucalyptus, vervain, peppermint, gum benzoin, basil, fennel, fir and balsam and mixtures thereof.

7. The wet wipe of claim 6, wherein the natural antibacterial agent comprises an extract from a plant selected from the group consisting of olive tree, rosemary, white and green tea, balm mint, chamomile, liquorice and mixtures thereof.

8. The wet wipe of claim 1, wherein the wet wipe is a baby wipe.

9. The wet wipe of claim 1, wherein the wet wipe is a feminine care hygiene wipe.

10. An article of commerce comprising the wet wipe of claim 1 packaged in a container, wherein the container is generally air-tight.
